SEQUENCE LISTING (100 sequences)

SEQ ID NO: 1 - DNA, 200 bp, Homo sapiens
acagttgcatcctctctgtactttgtcccctcataccatcagccaggcaggccagaaagccaaggaaagccaacgcctgaaaagatgatttacagcccttygtggctgagggaataacacataaagaactctttaaagctctcccactaggacgatccttcactataaacaccaggagcacacagagggtataaagcagc

SEQ ID NO: 2 - DNA, 200 bp, Homo sapiens
agcacagttccaagatgcgctgacatgttctgcaggcgagagagcctttcgctggactgtgactcacggaggtggtgaggccgctggttctaaccaagagratgccagcgagaccactcagcctcccctggtggctgccctggtttaatgctcatgaaatgccagcctgtggccattctgctgggtaaagagggtggtgt

SEQ ID NO: 3 - DNA, 161 bp, Homo sapiens
tggagaacccataggtaggaagcacctgggcctgaagacataaatagctggaggaggaatgmagccccctggactctccctgcacccctcctctgtgctcctctctgtccgtcctaatttttctctctcacagatgcctggtttctctgctccccagaccc

SEQ ID NO: 4 - DNA, 200 bp, Homo sapiens
atagcaggccagggctctgtaggcttgttttgctgctgtgcctgttgccaaatgcttgaaaatcctgaagtacaaacataacagtctgaattaaatttcaytgattttatctttctcttccttttctcactgtggtgcagttgttaatggaattctagccaaaaggatgtgggaggagaggaaggagccacaaagggaga

SEQ ID NO: 5 - DNA, 200 bp, Homo sapiens
tccaagcacaaaaacattagacttaagttcaagtggtaatatggaatgacatcagagtaccagccaaaagttgggaacaaaagacaacataaatgcgggtrtgaagtaacaaattctttattgaaggaattctttttagctccaacaattgataggttctattataatcaaaccctttatagcagattgatttcattaat

SEQ ID NO: 6 - DNA, 176 bp, Homo sapiens
gccagtaggttgattgtgaaagcactgtgcgcaacttccaggccatgtccttagaaagaccttaataggatatagayacaatggtgtaccatcttggatcttgcatgcagctgccttgcagtagtagaacaacaaggtggaagtttctctgtccctgtcatgaagaagcaccacca

SEQ ID NO: 7 - DNA, 151 bp, Homo sapiens
tgggccgcgtttctgtcgtcttgtgtttaggctgttatctgtgccctataaatatcaccaaaagcaatcgttaagtctgagccagatgaaccctgcagggstgccataacttaccatagactggatggatcaaacaccagaaatgtatttc

SEQ ID NO: 8 - DNA, 200 bp, Homo sapiens
aagaatggaaggacagcttgctacttctgggtgttcatcatgagcagttaaggctcaccatatggtctccactgatatttgggggaaggagacttgttacyacctaataatgagatgaaagatcctgattccctacttggtttactatgacaccgccctggcatgatggttcaggaaactctttacatttacagattgtc

SEQ ID NO: 9 - DNA, 200 bp, Homo sapiens
ttagcagtcccgcaaagcttgaggtttactcatttttactttctttagtctctgggcaaggaactaagaggtagtatagaattctagacgatggaaacttyagaatggacaggattttaaaaattatatattccattctcctgggttacaggtggtcttacagctaaactatttgagactgttgaagccattcgattttt

SEQ ID NO: 10 - DNA, 200 bp, Homo sapiens
tggacaatgggaataaagcatttctaaagcaccgactggagaggaaggcaacagagacaaggagagaagccgagagacatgtctgcgtgctgccacgcatytgagcgattgctctgtgaagagttgtacactgaacactttcaggggaggctgtttacccaggcaatgtcctcaaacaagcctgtgccggggtgtcctgg

SEQ ID NO: 11 - DNA, 151 bp, Homo sapiens
gattacaggcgtgagccacggcacctggagagaaaaaagcttctttaatgacgacctcaacataatttacatggagaagtttcttaggtggaaaaccgatytgtatggtcctcagaatgtaacctacaggttcttgccctcagaaaaatgc

SEQ ID NO: 12 - DNA, 200 bp, Homo sapiens
atagaatttttggtatactgaacaaagggattaaaaaggtcagttttatgtagggactttctttgctggggagggaaggtctcatccccaagaattatttmtttattttcccttcccagagtttttacatggaagtatgaaatattaccttcctactatataattaatagaaaaaataaacagagcccagattatactat

SEQ ID NO: 13 - DNA, 166 bp, Homo sapiens
gatgatacttagcttctgcgtcttcagtaaactcatctgtacaatggcctgttaaacctagctatttgaaaacatcagctaaaactaagtgatttttttgrtattcatttaggtcttttatttcttgatatgcttgaaatctaattgtcttttaaatgcactaata

SEQ ID NO: 14 - DNA, 200 bp, Homo sapiens
ccactgaaaatatcctctggtcatttatttggctcaacggacctatatggccattccctcttccacagttgtaaagactttgcctaatttctggcaactayagaagtagtggtaaggataatgtaaaatttctttttgtcttggaaattatcaaaccacttttgaagattgtattatctagtaatcagaaacttactttg

SEQ ID NO: 15 - DNA, 200 bp, Homo sapiens
tgtgcagcaatagtaactggaacaacatttctgcgatgggcagaagtggcaaggattgtaactcattagactcagaaggcgaggtcacctgagtaagcagkgtccttagtggaacagtgggtgtaagaggaaggagagatgggtgggtgtggggcttcttgccatctacatatgtagggtctgccttttacttgcacatc

SEQ ID NO: 16 - DNA, 200 bp, Homo sapiens
gcatagtactcagactcattgaaagtttatagaatttggcctgtgtggaaaactctgtgctccaagtacaacaactaactgaattctctaatttaaacaastttgaagggaagtgaaaggttataaaatgatacagactcataccgcagaatcaccttaaaatgcagctgttggagaaaaaaaaaaaggaagctttatgc

SEQ ID NO: 17 - DNA, 152 bp, Homo sapiens
aaggggcggcgctggccggactctctcgggtggactcccggagcggcggcggcggcggcggtggcagactgcgagtcacgtgcgacagaggaggcggagcrcggcggccgggtgggtggagaaaacaaagcccccagctgtcagcagaggaa

SEQ ID NO: 18 - DNA, 200 bp, Homo sapiens
attatagacaagcagagagggaatggatccgagggtgacctggctgaattaaaagtactgcatctaagtgtgtgcaggttgtagtccacagtaatggtgcyattcggagctgtgtgtacctgcactactgcatcaggtgaccttatccagcataacggtacaacagaaatttaacttgaaacaggtttattgacttgctt

SEQ ID NO: 19 - DNA, 200 bp, Homo sapiens
tctcccttcccccaacaccatgctaacagacatcaggcacacatggcagtggtgcctagccaggagaactgcaacaacagattattcacagggagcaacayaaagagtagacccaaaaccaccccgccagactcctaagccaggcagaatacaggcagtggggaaaaccctctggaaaagtagttcacaccctaaacaca

SEQ ID NO: 20 - DNA, 200 bp, Homo sapiens
caggtgctgggtggggaagtcttccttcactgcctggctggactcagaagccctccagtgcactgcagcagagaagactttgatctcaaagtaattttcaycttgatctcatctataggccctgaagactcttcctgggtgtctgtcaatgcaaggagtgggccttggcatttgttgtaacgaactgaaaaatgaccaca

SEQ ID NO: 21 - DNA, 200 bp, Homo sapiens
aggaaaatcaactagaaaataagtaaggacaaagcatttatttcactgtctaaagttcacaggggaaatatgagtataacaacaggaaatcaagatttttraaaagatagtagaagtacataggcttgcttaagtaaccaatcacaattctgaatgaagtacatttggatcgtaatttgatttaaagaaaaaaaaatgga

SEQ ID NO: 22 - DNA, 200 bp, Homo sapiens
aaattgagaattttaggctattgctataattgcctgtttggcttactcactagttagttctaagaaaaataaatgtagcccattaagatgtgcactgatgrattggattgaaaggtgctacctgagatattgttttttagggcaggctctcttggcctttttcaggatatgatatttttgataattttctgggacatacc

SEQ ID NO: 23 - DNA, 200 bp, Homo sapiens
ggagtaccatgagggttgctatttgttgtttttaacaactaatcaagagtgagtgaacaactatttataaactagatctcctatttttcagaatgctcttmtacgtataaatatgaaatgataaagatgtcaaatatctcagaggctatagctgggaacccgactgtgaaagtatgtgatatctgaacacatactagaaa

SEQ ID NO: 24 - DNA, 200 bp, Homo sapiens
actgggaactctggctccgaaaaactttgttatatatatcaaggatgttctggctttacattttatttattagctgtaaatacatgtgtggatgtgtaaayggagcttgtacatattggaaaggtcattgtggctatctgcatttataaatgtgtggtgctaactgtatgtgtctttatcagtgatggtctcacagagcc

SEQ ID NO: 25 - DNA, 176 bp, Homo sapiens
ttttcctgggcttcatgggccatttaaattgtgaacccccagggacacagtgcttagcccaagccatcagtgcttgaaaataggcatttctgtcatcgagwttctgtatgttgagatatattttgagataggcttaagggtgaggacatttggctggagtcagcaataagttgcaa

SEQ ID NO: 26 - DNA, 30 bp, Artificial Sequence
  primer
acgttggatgagccaacgcctgaaaagatg

SEQ ID NO: 27 - DNA, 30 bp, Artificial Sequence
  primer
acgttggatgagtgaaggatcgtcctagtg

SEQ ID NO: 28 - DNA, 18 bp, Artificial Sequence
  primer
agatgatttacagccctt

SEQ ID NO: 29 - DNA, 30 bp, Artificial Sequence
  primer
acgttggatggtgaggccgctggttctaac

SEQ ID NO: 30 - DNA, 30 bp, Artificial Sequence
  primer
acgttggatgtgagcattaaaccagggcag

SEQ ID NO: 31 - DNA, 20 bp, Artificial Sequence
  primer
ccgctggttctaaccaagag

SEQ ID NO: 32 - DNA, 30 bp, Artificial Sequence
  primer
acgttggatgggcctgaagacataaatagc

SEQ ID NO: 33 - DNA, 30 bp, Artificial Sequence
  primer
acgttggatgaattaggacggacagagagg

SEQ ID NO: 34 - DNA, 17 bp, Artificial Sequence
  primer
tagctggaggaggaatg

SEQ ID NO: 35 - DNA, 30 bp, Artificial Sequence
  primer
acgttggatgcacagtgagaaaaggaagag

SEQ ID NO: 36 - DNA, 30 bp, Artificial Sequence
  primer
acgttggatgtgcctgttgccaaatgcttg

SEQ ID NO: 37 - DNA, 22 bp, Artificial Sequence
  primer
aaggaagagaaagataaaatca

SEQ ID NO: 38 - DNA, 30 bp, Artificial Sequence
  primer
acgttggatgccagccaaaagttgggaaca

SEQ ID NO: 39 - DNA, 31 bp, Artificial Sequence
  primer
acgttggatggaacctatcaattgttggagc

SEQ ID NO: 40 - DNA, 20 bp, Artificial Sequence
  primer
aagacaacataaatgcgggt

SEQ ID NO: 41 - DNA, 30 bp, Artificial Sequence
  primer
acgttggatgagctgcatgcaagatccaag

SEQ ID NO: 42 - DNA, 30 bp, Artificial Sequence
  primer
acgttggatgcaggccatgtccttagaaag

SEQ ID NO: 43 - DNA, 20 bp, Artificial Sequence
  primer
ccaagatggtacaccattgt

SEQ ID NO: 44 - DNA, 30 bp, Artificial Sequence
  primer
acgttggatgggtgtttgatccatccagtc

SEQ ID NO: 45 - DNA, 30 bp, Artificial Sequence
  primer
acgttggatggcaatcgttaagtctgagcc

SEQ ID NO: 46 - DNA, 23 bp, Artificial Sequence
  primer
ccagtctatggtaagttatggca

SEQ ID NO: 47 - DNA, 30 bp, Artificial Sequence
  primer
acgttggatgccaagtagggaatcaggatc

SEQ ID NO: 48 - DNA, 30 bp, Artificial Sequence
  primer
acgttggatgggtctccactgatatttggg

SEQ ID NO: 49 - DNA, 24 bp, Artificial Sequence
  primer
ggatctttcatctcattattaggt

SEQ ID NO: 50 - DNA, 30 bp, Artificial Sequence
  primer
acgttggatgggcaaggaactaagaggtag

SEQ ID NO: 51 - DNA, 30 bp, Artificial Sequence
  primer
acgttggatgacctgtaacccaggagaatg

SEQ ID NO: 52 - DNA, 18 bp, Artificial Sequence
  primer
tctagacgatggaaactt

SEQ ID NO: 53 - DNA, 30 bp, Artificial Sequence
  primer
acgttggatgcagtgtacaactcttcacag

SEQ ID NO: 54 - DNA, 30 bp, Artificial Sequence
  primer
acgttggatgacagagacaaggagagaagc

SEQ ID NO: 55 - DNA, 20 bp, Artificial Sequence
  primer
cttcacagagcaatcgctca

SEQ ID NO: 56 - DNA, 30 bp, Artificial Sequence
  primer
acgttggatgtttctgagggcaagaacctg

SEQ ID NO: 57 - DNA, 30 bp, Artificial Sequence
  primer
acgttggatgtggagaagtttcttaggtgg

SEQ ID NO: 58 - DNA, 21 bp, Artificial Sequence
  primer
ttacattctgaggaccataca

SEQ ID NO: 59 - DNA, 30 bp, Artificial Sequence
  primer
acgttggatggtagggactttctttgctgg

SEQ ID NO: 60 - DNA, 30 bp, Artificial Sequence
  primer
acgttggatgtgtaaaaactctgggaaggg

SEQ ID NO: 61 - DNA, 20 bp, Artificial Sequence
  primer
ctcatccccaagaattattt

SEQ ID NO: 62 - DNA, 30 bp, Artificial Sequence
  primer
acgttggatgaatggcctgttaaacctagc

SEQ ID NO: 63 - DNA, 32 bp, Artificial Sequence
  primer
acgttggatggcatatcaagaaataaaagacc

SEQ ID NO: 64 - DNA, 23 bp, Artificial Sequence
  primer
gctaaaactaagtgatttttttg

SEQ ID NO: 65 - DNA, 31 bp, Artificial Sequence
  primer
acgttggatgcacagttgtaaagactttgcc

SEQ ID NO: 66 - DNA, 30 bp, Artificial Sequence
  primer
acgttggatggtggtttgataatttccaag

SEQ ID NO: 67 - DNA, 20 bp, Artificial Sequence
  primer
tgcctaatttctggcaacta

SEQ ID NO: 68 - DNA, 30 bp, Artificial Sequence
  primer
acgttggatgctcattagactcagaaggcg

SEQ ID NO: 69 - DNA, 30 bp, Artificial Sequence
  primer
acgttggatgccatctctccttcctcttac

SEQ ID NO: 70 - DNA, 19 bp, Artificial Sequence
  primer
aggtcacctgagtaagcag

SEQ ID NO: 71 - DNA, 30 bp, Artificial Sequence
  primer
acgttggatgctgcggtatgagtctgtatc

SEQ ID NO: 72 - DNA, 30 bp, Artificial Sequence
  primer
acgttggatgctctgtgctccaagtacaac

SEQ ID NO: 73 - DNA, 21 bp, Artificial Sequence
  primer
aacctttcacttcccttcaaa

SEQ ID NO: 74 - DNA, 30 bp, Artificial Sequence
  primer
acgttggatgagactgcgagtcacgtgcga

SEQ ID NO: 75 - DNA, 30 bp, Artificial Sequence
  primer
acgttggatggctttgttttctccacccac

SEQ ID NO: 76 - DNA, 17 bp, Artificial Sequence
  primer
gacagaggaggcggagc

SEQ ID NO: 77 - DNA, 30 bp, Artificial Sequence
  primer
acgttggatgtggataaggtcacctgatgc

SEQ ID NO: 78 - DNA, 30 bp, Artificial Sequence
  primer
acgttggatgagtgtgtgcaggttgtagtc

SEQ ID NO: 79 - DNA, 17 bp, Artificial Sequence
  primer
tacacacagctccgaat

SEQ ID NO: 80 - DNA, 30 bp, Artificial Sequence
  primer
acgttggatgctgtattctgcctggcttag

SEQ ID NO: 81 - DNA, 30 bp, Artificial Sequence
  primer
acgttggatggccaggagaactgcaacaac

SEQ ID NO: 82 - DNA, 18 bp, Artificial Sequence
  primer
gttttgggtctactcttt

SEQ ID NO: 83 - DNA, 30 bp, Artificial Sequence
  primer
acgttggatggtcttcagggcctatagatg

SEQ ID NO: 84 - DNA, 30 bp, Artificial Sequence
  primer
acgttggatgagtgcactgcagcagagaag

SEQ ID NO: 85 - DNA, 23 bp, Artificial Sequence
  primer
cagggcctatagatgagatcaag

SEQ ID NO: 86 - DNA, 30 bp, Artificial Sequence
  primer
acgttggatggcaagcctatgtacttctac

SEQ ID NO: 87 - DNA, 30 bp, Artificial Sequence
  primer
acgttggatgactgtctaaagttcacaggg

SEQ ID NO: 88 - DNA, 23 bp, Artificial Sequence
  primer
cctatgtacttctactatctttt

SEQ ID NO: 89 - DNA, 30 bp, Artificial Sequence
  primer
acgttggatgtgcctgtttggcttactcac

SEQ ID NO: 90 - DNA, 30 bp, Artificial Sequence
  primer
acgttggatgtatctcaggtagcacctttc

SEQ ID NO: 91 - DNA, 20 bp, Artificial Sequence
  primer
cattaagatgtgcactgatg

SEQ ID NO: 92 - DNA, 30 bp, Artificial Sequence
  primer
acgttggatgctagatctcctatttttcag

SEQ ID NO: 93 - DNA, 30 bp, Artificial Sequence
  primer
acgttggatggttcccagctatagcctctg

SEQ ID NO: 94 - DNA, 23 bp, Artificial Sequence
  primer
ctcctatttttcagaatgctctt

SEQ ID NO: 95 - DNA, 30 bp, Artificial Sequence
  primer
acgttggatgtgcagatagccacaatgacc

SEQ ID NO: 96 - DNA, 30 bp, Artificial Sequence
  primer
acgttggatgagctgtaaatacatgtgtgg

SEQ ID NO: 97 - DNA, 19 bp, Artificial Sequence
  primer
tccaatatgtacaagctcc

SEQ ID NO: 98 - DNA, 30 bp, Artificial Sequence
  primer
acgttggatgttagcccaagccatcagtgc

SEQ ID NO: 99 - DNA, 30 bp, Artificial Sequence
  primer
acgttggatgctcacccttaagcctatctc

SEQ ID NO: 100 - DNA, 17 bp, Artificial Sequence
  primer
gcatttctgtcatcgag

(19) United States
(12) Patent Application Publication
(10) Pub. No.: US 2006/0263817 A1
(43) Pub. Date: Nov. 23, 2006

(54) Genetic polymorphism associated with myocardial infarction and uses thereof

(76) Inventors: Kyu-sang Lee, Suwon-si (KR); Ki-eun Kim, Seoul (KR); Byung-chul Kim, Hwaseong-si (KR); Kyung-Hee Park, Seoul (KR)

Correspondence Address: CANTOR COLBURN, LLP, 55 GRIFFIN ROAD SOUTH, BLOOMFIELD, CT, 06002, US

(21) Appl. No.: 11/438,614

(22) Filed: May 22, 2006

(30) Foreign Application Priority Data
May 21, 2005 (KR) 10-2005-0042783

(51) Int. Cl. C12Q 1/68; G01N 33/53; C07H 21/04; C07K 16/40

(52) U.S. Cl. 435/6; 435/7.1; 530/388.1; 536/23.2

(57) ABSTRACT

A genetic polymorphism associated with myocardial infarction is provided. More particularly, a polynucleotide including a single nucleotide polymorphism (SNP) associated with myocardial infarction, a complementary polynucleotide of the nucleotide sequences, a polynucleotide hybridized with one of the polynucleotides, a polypeptide encoded by one of the polynucleotides, an antibody bound to the polypeptide, a microarray and a kit including the polynucleotides, a myocardial infarction diagnosis method, a SNP detecting method and a method of screening pharmaceutical compositions for myocardial infarction are provided.

### CROSS-REFERENCE TO RELATED PATENT APPLICATION

This application claims the benefit of Korean Patent Application NO: 10-2005-0042783, filed on May 21, 2005, in the Korean Intellectual Property Office, the disclosure of which is incorporated herein in its entirety by reference.

### BACKGROUND OF THE INVENTION

1. Field of the Invention

The present invention relates to a genetic polymorphism associated with myocardial infarction and uses thereof.

2. Description of the Related Art

In all humans, 99.9% of the base sequences of the human genome are identical. Diversity in individuals' appearance, behavior and susceptibility to certain diseases is caused by partial differences in the remaining 0.1% of the base sequences in the human genome. That is, there are about 3 billion base pairs in the human genome, and differences in about 3 million base sequences of the human genome influence the diversity among individuals, communities, races and peoples. The differences in base sequences are a cause of the differences in disease distributions as well as other phenotypic distinctions such as skin color for different races. Polymorphic single base pairs are positioned in the human genome at intervals of about 1.0 kb, on average. Variation across a population in the sequence at a single base pair is referred to as a Single Nucleotide Polymorphism (SNP). Causes of the diversity among individuals and communities or the difference between a disease group and a normal group may be found through the analysis of the 3 million SNPs, without analyzing the entire human base sequence.

A prime object of genetics is to map phenotypic differences, such as susceptibility to diseases in humans, to variations in DNA. A polymorphic marker existing in all genomes is the best means of obtaining this object. Among polymorphic markers, microsatellite markers have been commonly used to distinguish individuals and find genes related to a genetic disease having family history, but SNPs have drawn attention with the development of DNA chips. Automated SNP detection on a large scale is possible because of the high frequency of SNPs, the stability of SNPs, and the even distribution of SNPs across all genomes. SNPs will contribute to 21^{st} century predictive medical science by permitting prediction of diseases or disease risk of individuals and investigation of individuals' reactions to therapeutics when used in conjunction with up-to-date biotechnology, such as a DNA chip technique or a high speed DNA sequence analysis technique.

A study of SNPs involves analysis of genotypes and their distribution throughout a population. If presence of a disease is significantly associated with a genotype, the relationship between the genotype and the disease can be established. In most studies of SNPs, if a single genotype or several genotypes have significantly different distributions in a disease group vs. a normal group, the differences in disease frequency according to the genotype may be analyzed.

About 510,000 SNPs, approximately one sixth of the 3 million SNPs in human genomes, occur in genes. It is important to know the distribution of such mutations in genes because the mutations are directly related to gene expression or protein functions. If a genotype associated with a certain disease can effect a change in gene expression or protein function, the gene or the protein is likely to be a cause of the disease. In this case, the gene can be a target gene for disease detection and treatment. The susceptibility to the disease may also be analyzed using SNP analysis of the gene.

SNPs in transcription regulatory regions of a base sequence, such as a promoter, can regulate the quantity of expressed genes. On rare occasions, SNPs also influence the stability and translation efficiency of RNA when located in sequences at exon-intron boundaries, affecting RNA splicing, or in a 3′-untranslated region (3′-UTR). Although SNPs located in a noncoding region or in a transcription and translation regulatory region do not affect proteins directly, such SNPs may still be found to be associated with a disease and may be a useful index for determining susceptibility to the disease.

Cardiovascular disease is a major cause of death in industrialized countries around the world, and has been a major cause of death in the Republic of Korea since the 1970s. According to the Korea National Statistical Office, in 2003, 22,000 out of 246,000 deaths (9,087 per 100,000, or 9.1%) were the result of cardiac disorder and hyperpiesia, which are the third leading cause of death in Korea following cancer and cerebrovascular disease.

Coronary artery disease, which ranks high among cardiovascular diseases, is usually caused by arteriosclerosis, the blocking or narrowing of coronary arteries supplying blood to the heart. Blocking of the coronary artery indicates myocardial infarction and narrowing of the coronary artery indicates angina pectoris. The causes of and risk factors for coronary artery disease are known to be hyperlipidemia (hypercholesterolemia), hyperpiesia, smoking, diabetes, genetic inheritance, obesity, lack of exercise, stress and menopause. A subject having multiple risk factors for coronary artery disease has a higher risk of incidence.

The most serious problem in early diagnosis or prognosis of various cardiovascular diseases and associated diseases, including myocardial infarction, is that the diagnosis or prediction can be performed using a physical technique only when the diseases are at an advanced stage. Currently, X-ray and ultrasonography of the interior of the heart and coronary artery can be used for cardiovascular disease diagnosis, but this diagnosis is only possible at an advanced stage. However, the development of various recent molecular biological techniques and the preliminary completion of the human genome project enable the finding of genes or genetic variations directly or indirectly related to a disease. Therefore, early diagnosis of cardiovascular disease using a genetic factor, instead of using a conventional diagnostic method depending on a phenotypic or physical characteristic of the disease, has become available.

However, predicting the incidence of cardiovascular disease using only a genetic factor is difficult since the occurrence of cardiovascular disease may be affected by various environmental and habitual factors.

### SUMMARY OF THE INVENTION

The present inventors found SNPs associated with myocardial infarction in a Korean population, which make it possible to predict the incidence, probability of, and genetic susceptibility to myocardial infarction according to environmental and habitual factors.

According to an aspect of the present invention, there is provided a polynucleotide comprising at least 8 contiguous nucleotides of a nucleotide sequence selected from sequence identification number (SEQ ID NO:) 3, SEQ ID NOS: 5 to 7 and SEQ ID NOS: 19 to 24, wherein the at least 8 contiguous nucleotides comprise a base at a single nucleotides polymorphism (SNP) position in the selected nucleotide sequence, wherein the SNPs are positioned at the 62^{nd} nucleotide in SEQ ID NO: 3, at the 77^{th} nucleotide in SEQ ID NO: 6 and at the 101^{st} nucleotide in SEQ ID NOS: 5,7 and 19 to 24. Additionally, the complement of such a polynucleotide is provided.

According to another aspect of the present invention, there is provided a polynucleotide specifically hybridized with the polynucleotide, or the complement of the hybridized polynucleotide.

According to another aspect of the present invention, there is provided a polypeptide encoded by the polynucleotide.

According to another aspect of the present invention, there is provided an antibody, which specifically binds to the polypeptide.

According to another aspect of the present invention, there is provided a microarray for detecting a SNP. The microarray comprises the polynucleotide, the polypeptide encoded by the polynucleotide or the cDNA thereof.

According to another aspect of the present invention, there is provided a kit for detecting a SNP. The kit comprises the polynucleotide, the polypeptide encoded by the polynucleotide or the cDNA thereof.

According to another aspect of the present invention, there is provided a method of identifying a risk of incidence of myocardial infarction for a subject. The method comprises determining an allele present in the subject at a single nucleotide polymorphism (SNP). The SNP is identified by a polymorphic sequence selected from the group consisting of SEQ ID NO:1-25, wherein the SNPs are positioned at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25. In another embodiment, the SNP is identified by a polymorphic sequence selected from the group consisting of SEQ ID NO: 3, SEQ ID NOS: 5 to 7 and SEQ ID NOS: 19 to 24.

In another embodiment, the method comprises determining an allele present in the subject at a SNP, wherein if the subject is aged 55 and older, then the SNP is identified by polymorphic sequence SEQ ID NO: 1 or SEQ ID NO: 2; wherein if the subject is aged 54 and younger, then the SNP is identified by polymorphic sequence SEQ ID NO: 3 or SEQ ID NO: 4; wherein if the subject is non-smoking, then the SNP is identified by a polymorphic sequence selected from the group consisting of SEQ ID NO: 4 to SEQ ID NO: 8; wherein if the subject is male, then the SNP is identified by a polymorphic sequence selected from the group consisting of SEQ ID NO: 4 and SEQ ID NO: 8 to SEQ ID NO: 11; wherein if the subject does not having a family history of hyperpiesia, then the SNP is identified by polymorphic sequence SEQ ID NO: 12 or SEQ ID NO: 13; wherein if the subject has hyperpiesia, then the SNP is identified by polymorphic sequence SEQ ID NO: 14; wherein if the subject doesnot have hyperpiesia, then the SNP is identified by a polymorphic sequence selected from the group consisting of SEQ ID NO: 8 to SEQ ID NO: 10 and SEQ ID NO: 5 to SEQ ID NO: 21; wherein if the subject has a family history of diabetes, then the SNP is identified by polymorphic sequence SEQ ID NO: 22; wherein if the subject does not have a family history of diabetes, then the SNP is identified by a polymorphic sequence selected from the group consisting of SEQ ID NO: 4, SEQ ID NO: 8 to SEQ ID NO: 10, SEQ ID NO: 23 and SEQ ID NO: 24; or wherein if the subject has a high CRP level, then the SNP is identified by polymorphic sequence SEQ ID NO: 25.

According to another aspect of the present invention, there is provided a method of detecting a SNP in nucleic acid molecules including contacting a test sample containing nucleic acid molecules with a polynucleotide comprising at least 8 contiguous nucleotides of a polymorphic sequence selected from the group consisting of nucleotide sequences SEQ ID NO: 1-25, wherein the at least 8 contiguous nucleotides comprise a base at a single nucleotide polymorphism (SNP) position in the selected polymorphic sequence, wherein the SNPs are positioned at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25, or the complement thereof, under strict hybridization conditions such that specific hybridization between nucleic acid molecules in the test sample and the polynucleotide can occur; and detecting the formation of a hybridized double-strand.

According to another aspect of the present invention, there is provided a method of screening pharmaceutical compositions for myocardial infarction including contacting a candidate material with a polypeptide encoded by the polynucleotide comprising at least 8 contiguous nucleotides of a nucleotide sequence selected from sequence identification number (SEQ ID NO:) 3, SEQ ID NOS: 5 to 7 and SEQ ID NOS: 19 to 24, wherein the at least 8 contiguous nucleotides comprise a base at a single nucleotides polymorphism (SNP) position in the selected nucleotide sequence under proper conditions for formation of a binding complex, and detecting the formation of the binding complex between the polypeptide and the candidate material.

The above aspects and advantages of the present invention will become more apparent by describing in detail exemplary embodiments thereof.

### DETAILED DESCRIPTION OF THE INVENTION

The association of certain single nucleotide polymorphisms (SNPs) with the presence or risk of myocardial infarction in a Korean population was discovered.

A single nucleotide polymorphism (SNP) associated with myocardial infarction according to an aspect of the present invention can be identified by a polymorphic sequence comprising nucleotide sequences SEQ ID NO: 1-25. In these polymorphic sequences, the SNPs are positioned at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25. Herein, a “polymorphic sequence” is a polynucleotide sequence including a polymorphic site at which a SNP occurs as well as sequence flanking the polymorphic site. The polymorphic sequence can be used for identification of the SNP in a nucleic acid. All or only part of the polymorphic sequence flanking the polymorphic site can be used by the skilled practitioner to identify the SNP in a nucleic acid. The nucleic acid may be DNA or RNA.

The present invention also provides Korean-specific SNPs associated with myocardial infarction.

A Korean-specific SNP associated with myocardial infarction according to an aspect of the present invention can be identified by a polymorphic sequence comprising nucleotide sequences SEQ ID NO: 3, SEQ ID NOS: 5 to 7 and SEQ ID NOS: 19 to 24. The SNPs are positioned in these polymorphic sequences at the 62^{nd} nucleotide in SEQ ID NO: 3, at the 77^{th} nucleotide in SEQ ID NO: 6 and at the 101^{st} nucleotide in SEQ ID NOS: 5, 7 and 19 to 24.

In the Examples of the present invention, a series of selections were made in order to find SNPs closely associated with cardiovascular disease, more particularly with myocardial infarction. DNA was isolated from blood of a patient group having myocardial infarction and a normal group and then amplified. After an analysis of the SNP alleles in the DNA, SNPs for which the genotype had very different appearance frequencies in the patient group the normal group were identified. The SNPs, and the genotype thereof, identified in the Examples are described below in Table 1.

The SNPs in Table 1 are positioned in their respective reference polymorphic sequences at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25.

TABLE 1

| | | | | | | | |
|---|---|---|---|---|---|---|---|
| | | | SEQ ID | | | | |
| NO: | strata | alias_id | NO: | gene name | SNP_function | AA_change | AA_position |

| | | | | | | | |
|---|---|---|---|---|---|---|---|
| 1 | high age | MI_0458 | 1 | intergenic | intergenic | n/a | n/a |
| 2 | high age | MI_0720 | 2 | LGALS2 | intron | null | null |
| 3 | low age | MI_0524 | 3 | intergenic | intergenic | n/a | n/a |
| 4 | low age | MI_1186 | 4 | intergenic | intergenic | n/a | n/a |
| 5 | non | MI_0125 | 5 | intergenic | intergenic | n/a | n/a |
| | smoking | | | | | | |
| 6 | non | MI_1052 | 6 | intergenic | intergenic | n/a | n/a |
| | smoking | | | | | | |
| 7 | non | MI_1186 | 4 | intergenic | intergenic | n/a | n/a |
| | smoking | | | | | | |
| 8 | non | MI_0300 | 7 | intergenic | intergenic | n/a | n/a |
| | smoking | | | | | | |
| 9 | non | MI_0042 | 8 | intergenic | intergenic | n/a | n/a |
| | smoking | | | | | | |
| 10 | smoking | none | | | | | |
| 11 | male | MI_0393 | 9 | MANBA | intron | null | null |
| 12 | male | MI_0370 | 10 | MANBA | mrna-utr | null | null |
| 13 | male | MI_1186 | 4 | intergenic | intergenic | n/a | n/a |
| 14 | male | MI_0299 | 11 | intergenic | intergenic | n/a | n/a |
| 15 | male | MI_0042 | 8 | intergenic | intergenic | n/a | n/a |
| 16 | no HTNF | MI_1273 | 12 | intergenic | intergenic | n/a | n/a |
| 17 | no HTNF | MI_0294 | 13 | intergenic | intergenic | n/a | n/a |
| 18 | with HTNF | none | | | | | |
| 19 | with HTN | MI_0100 | 14 | intergenic | intergenic | n/a | n/a |
| 20 | w/o HTN | MI_0292 | 15 | DSCR1 | intron | null | null |
| 21 | w/o HTN | MI_0393 | 9 | MANBA | intron | null | null |
| 22 | w/o HTN | MI_0370 | 10 | MANBA | mrna-utr | null | null |
| 23 | w/o HTN | MI_1329 | 16 | NFKB1 | intron | null | null |
| 24 | w/o HTN | MI_0177 | 17 | intergenic | intergenic | n/a | n/a |
| 25 | w/o HTN | MI_1096 | 18 | intergenic | intergenic | n/a | n/a |
| 26 | w/o HTN | MI_0042 | 8 | intergenic | intergenic | n/a | n/a |
| 27 | w/o HTN | MI_0009 | 19 | intergenic | intergenic | n/a | n/a |
| 28 | w/o HTN | MI_0228 | 20 | intergenic | intergenic | n/a | n/a |
| 29 | w/o HTN | MI_0233 | 21 | intergenic | intergenic | n/a | n/a |
| 30 | with DMF | MI_1001 | 22 | ITPR2 | intron | null | null |
| 31 | w/o DMF | MI_0783 | 23 | LPL | mrna-utr | null | null |
| 32 | w/o DMF | MI_0567 | 24 | LPL | mrna-utr | null | null |
| 33 | w/o DM | MI_0292 | 15 | DSCR1 | intron | null | null |
| 34 | w/o DM | MI_0393 | 9 | MANBA | intron | null | null |
| 35 | w/o DM | MI_1186 | 4 | intergenic | intergenic | n/a | n/a |
| 36 | w/o DM | MI_0370 | 10 | MANBA | mrna-utr | null | null |
| 37 | w/o DM | MI_0042 | 8 | intergenic | intergenic | n/a | n/a |
| 38 | with DM | none | | | | | |
| 39 | high CRP | MI_1363 | 25 | intergenic | intergenic | n/a | n/a |
| 40 | low CRP | none | | | | | |

| | | | | | | | | | | | | |
|---|---|---|---|---|---|---|---|---|---|---|---|---|
| | cas_{—} | con_{—} | allele | allele | cas_{—} | cas_{—} | cas_{—} | con_{—} | con_{—} | con_{—} | case | control |
| NO: | num | num | A | a | AA | Aa | aa | AA | Aa | aa | MAF | MAF |

| | | | | | | | | | | | | |
|---|---|---|---|---|---|---|---|---|---|---|---|---|
| 1 | 114 | 104 | C | T | 96 | 15 | 3 | 58 | 44 | 2 | 0.91 | 0.77 |
| 2 | 115 | 104 | A | G | 3 | 32 | 80 | 15 | 37 | 52 | 0.17 | 0.32 |
| 3 | 101 | 84 | C | A | 95 | 6 | 0 | 65 | 19 | 0 | 0.97 | 0.89 |
| 4 | 103 | 87 | T | C | 70 | 30 | 3 | 43 | 32 | 12 | 0.83 | 0.68 |
| 5 | 32 | 62 | A | G | 18 | 11 | 3 | 14 | 33 | 15 | 0.73 | 0.49 |
| 6 | 32 | 62 | T | C | 7 | 17 | 8 | 31 | 27 | 4 | 0.48 | 0.72 |
| 7 | 32 | 61 | T | C | 26 | 5 | 1 | 32 | 22 | 7 | 0.89 | 0.70 |
| 8 | 32 | 61 | G | C | 1 | 19 | 12 | 15 | 30 | 16 | 0.33 | 0.49 |
| 9 | 31 | 62 | T | C | 0 | 3 | 28 | 4 | 15 | 43 | 0.05 | 0.19 |
| 10 | | | | | | | | | | | | |
| 11 | 221 | 190 | C | T | 50 | 124 | 47 | 31 | 83 | 76 | 0.51 | 0.38 |
| 12 | 221 | 192 | T | C | 44 | 119 | 58 | 73 | 84 | 35 | 0.47 | 0.60 |
| 13 | 218 | 189 | T | C | 149 | 63 | 6 | 97 | 74 | 18 | 0.83 | 0.71 |
| 14 | 219 | 192 | T | C | 36 | 109 | 74 | 62 | 85 | 45 | 0.41 | 0.54 |
| 15 | 212 | 185 | T | C | 2 | 44 | 166 | 10 | 50 | 125 | 0.11 | 0.19 |
| 16 | 141 | 67 | C | A | 120 | 20 | 1 | 46 | 15 | 6 | 0.92 | 0.80 |
| 17 | 141 | 67 | A | G | 81 | 59 | 1 | 32 | 28 | 7 | 0.78 | 0.69 |
| 18 | | | | | | | | | | | | |
| 19 | 83 | 25 | C | T | 65 | 18 | 0 | 16 | 5 | 4 | 0.89 | 0.74 |
| 20 | 126 | 149 | T | G | 9 | 65 | 52 | 4 | 45 | 100 | 0.33 | 0.18 |
| 21 | 131 | 149 | C | T | 35 | 71 | 25 | 23 | 66 | 60 | 0.54 | 0.38 |
| 22 | 131 | 150 | T | C | 23 | 69 | 39 | 56 | 67 | 27 | 0.44 | 0.60 |
| 23 | 131 | 149 | C | G | 37 | 68 | 26 | 72 | 62 | 15 | 0.54 | 0.69 |
| 24 | 129 | 149 | A | G | 0 | 5 | 124 | 0 | 20 | 129 | 0.02 | 0.07 |
| 25 | 131 | 150 | T | C | 0 | 2 | 129 | 0 | 13 | 137 | 0.01 | 0.04 |
| 26 | 124 | 144 | T | C | 1 | 27 | 96 | 10 | 41 | 93 | 0.12 | 0.21 |
| 27 | 130 | 150 | T | C | 1 | 51 | 78 | 9 | 41 | 100 | 0.20 | 0.20 |
| 28 | 129 | 147 | T | C | 1 | 47 | 81 | 12 | 50 | 85 | 0.19 | 0.25 |
| 29 | 129 | 147 | G | A | 5 | 62 | 62 | 20 | 66 | 61 | 0.28 | 0.36 |
| 30 | 44 | 8 | A | G | 44 | 0 | 0 | 6 | 2 | 0 | 1.00 | 0.88 |
| 31 | 168 | 61 | C | A | 122 | 46 | 0 | 49 | 9 | 3 | 0.86 | 0.88 |
| 32 | 168 | 62 | T | C | 124 | 44 | 0 | 50 | 9 | 3 | 0.87 | 0.88 |
| 33 | 211 | 165 | T | G | 16 | 102 | 93 | 4 | 47 | 114 | 0.32 | 0.17 |
| 34 | 221 | 165 | C | T | 50 | 124 | 47 | 26 | 70 | 69 | 0.51 | 0.37 |
| 35 | 218 | 165 | T | C | 149 | 63 | 6 | 83 | 65 | 17 | 0.83 | 0.70 |
| 36 | 221 | 167 | T | C | 44 | 119 | 58 | 64 | 74 | 29 | 0.47 | 0.60 |
| 37 | 212 | 161 | T | C | 2 | 44 | 166 | 9 | 43 | 109 | 0.11 | 0.19 |
| 38 | | | | | | | | | | | | |
| 39 | 43 | 32 | T | A | 26 | 17 | 0 | 13 | 13 | 6 | 0.80 | 0.61 |
| 40 | | | | | | | | | | | | |

| | | | | | | | |
|---|---|---|---|---|---|---|---|
| | genotype | allelic | | | | | |
| | Fisher's | Fisher's | | | | | |
| | exact test | exact test | | allel_OR_{—} | allel_OR_{—} | | |
| NO: | p-value | p-value | allel_OR | LB | UB | allel_assoc | allel_power |

| | | | | | | | |
|---|---|---|---|---|---|---|---|
| 1 | 0.000002 | 0.000075 | 2.96 | 1.70 | 5.14 | R | 69.4% |
| 2 | 0.000972 | 0.000134 | 0.42 | 0.26 | 0.66 | P | 58.1% |
| 3 | 0.001100 | 0.001629 | 4.17 | 1.62 | 10.69 | R | 59.4% |
| 4 | 0.005181 | 0.001117 | 2.24 | 1.39 | 3.62 | R | 43.3% |
| 5 | 0.004635 | 0.001776 | 2.86 | 1.48 | 5.51 | R | 61.7% |
| 6 | 0.005617 | 0.002253 | 0.37 | 0.20 | 0.69 | P | 50.4% |
| 7 | 0.021910 | 0.005554 | 3.41 | 1.42 | 8.19 | R | 61.4% |
| 8 | 0.022718 | 0.042748 | 0.50 | 0.27 | 0.95 | P | 30.7% |
| 9 | 0.069440 | 0.012514 | 0.22 | 0.06 | 0.78 | P | 58.2% |
| 10 | | | | | | | |
| 11 | 0.000191 | 0.000337 | 1.67 | 1.26 | 2.20 | R | 37.0% |
| 12 | 0.000204 | 0.000209 | 0.59 | 0.45 | 0.78 | P | 40.6% |
| 13 | 0.000324 | 0.000056 | 1.98 | 1.42 | 2.76 | R | 58.0% |
| 14 | 0.000495 | 0.000204 | 0.59 | 0.45 | 0.78 | P | 42.6% |
| 15 | 0.007264 | 0.003599 | 0.55 | 0.37 | 0.81 | P | 37.5% |
| 16 | 0.002205 | 0.000512 | 2.98 | 1.63 | 5.47 | R | 38.2% |
| 17 | 0.003685 | 0.038639 | 1.65 | 1.04 | 2.63 | R | 11.7% |
| 18 | | | | | | | |
| 19 | 0.003822 | 0.011157 | 2.89 | 1.30 | 6.42 | R | 13.5% |
| 20 | 0.000056 | 0.000046 | 2.27 | 1.53 | 3.37 | R | 48.2% |
| 21 | 0.000301 | 0.000129 | 1.94 | 1.38 | 2.71 | R | 52.2% |
| 22 | 0.000549 | 0.000198 | 0.53 | 0.38 | 0.74 | P | 50.9% |
| 23 | 0.001204 | 0.000337 | 0.53 | 0.37 | 0.75 | P | 45.0% |
| 24 | 0.005887 | 0.007102 | 0.27 | 0.10 | 0.74 | P | 53.5% |
| 25 | 0.007727 | 0.008512 | 0.17 | 0.04 | 0.76 | P | 58.3% |
| 26 | 0.010581 | 0.003689 | 0.49 | 0.31 | 0.80 | P | 45.5% |
| 27 | 0.010709 | 0.833162 | 1.05 | 0.69 | 1.58 | N | 3.2% |
| 28 | 0.011201 | 0.100770 | 0.70 | 0.46 | 1.05 | N | 16.7% |
| 29 | 0.016238 | 0.044854 | 0.69 | 0.48 | 0.99 | P | 20.7% |
| 30 | 0.021116 | 0.022405 | 30.52 | 1.39 | 668.5 | R | 50.6% |
| 31 | 0.004233 | 0.757756 | 0.88 | 0.47 | 1.65 | N | 3.1% |
| 32 | 0.004687 | 0.875624 | 0.91 | 0.49 | 1.71 | N | 3.0% |
| 33 | 0.000004 | 0.000002 | 2.33 | 1.63 | 3.32 | R | 40.9% |
| 34 | 0.000080 | 0.000187 | 1.75 | 1.31 | 2.34 | R | 35.1% |
| 35 | 0.000196 | 0.000039 | 2.06 | 1.46 | 2.91 | R | 55.8% |
| 36 | 0.000263 | 0.000208 | 0.58 | 0.43 | 0.77 | P | 35.9% |
| 37 | 0.009124 | 0.004571 | 0.55 | 0.36 | 0.82 | P | 31.9% |
| 38 | | | | | | | |
| 39 | 0.006732 | 0.010646 | 2.60 | 1.25 | 5.40 | R | 25.8% |
| 40 | | | | | | | |

| | | | | | | | | | | |
|---|---|---|---|---|---|---|---|---|---|---|
| | domi_{—} | domi_{—} | domi_{—} | domi_{—} | domi_{—} | rece_{—} | rece_{—} | rece_{—} | rece_{—} | rece_{—} |
| NO: | OR | OR_LB | OR_UB | assoc | power | OR | OR_LB | OR_UB | assoc | power |

| | | | | | | | | | | |
|---|---|---|---|---|---|---|---|---|---|---|
| 1 | 0.73 | 0.12 | 4.43 | N | 3.5% | 4.23 | 2.24 | 7.98 | R | 79.0% |
| 2 | 0.44 | 0.25 | 0.76 | P | 32.3% | 0.16 | 0.04 | 0.57 | P | 68.5% |
| 3 | 1.20 | 0.02 | 61.18 | N | 2.8% | 4.37 | 1.71 | 11.22 | R | 58.0% |
| 4 | 5.33 | 1.45 | 19.57 | R | 52.3% | 2.17 | 1.20 | 3.92 | R | 24.1% |
| 5 | 3.09 | 0.82 | 11.59 | N | 27.8% | 4.41 | 1.76 | 11.04 | R | 50.4% |
| 6 | 0.21 | 0.06 | 0.75 | P | 20.9% | 0.28 | 0.11 | 0.74 | P | 48.8% |
| 7 | 4.02 | 0.47 | 34.20 | N | 21.1% | 3.93 | 1.42 | 10.89 | R | 53.1% |
| 8 | 0.59 | 0.24 | 1.48 | N | 10.4% | 0.10 | 0.01 | 0.79 | P | 65.9% |
| 9 | 0.24 | 0.07 | 0.90 | P | 44.8% | 0.21 | 0.01 | 3.96 | N | 17.2% |
| 10 | | | | | | | | | | |
| 11 | 2.47 | 1.60 | 3.81 | R | 59.5% | 1.50 | 0.91 | 2.47 | N | 9.9% |
| 12 | 0.63 | 0.39 | 1.01 | N | 12.6% | 0.41 | 0.26 | 0.63 | P | 59.6% |
| 13 | 3.72 | 1.44 | 9.58 | R | 51.0% | 2.05 | 1.37 | 3.07 | R | 40.8% |
| 14 | 0.60 | 0.39 | 0.93 | P | 16.6% | 0.41 | 0.26 | 0.66 | P | 55.4% |
| 15 | 0.58 | 0.37 | 0.90 | P | 23.6% | 0.17 | 0.04 | 0.77 | P | 52.1% |
| 16 | 13.77 | 1.62 | 116.84 | R | 64.7% | 2.61 | 1.30 | 5.22 | R | 20.1% |
| 17 | 16.33 | 1.97 | 135.67 | R | 75.9% | 1.48 | 0.82 | 2.65 | N | 6.3% |
| 18 | | | | | | | | | | |
| 19 | 34.95 | 1.81 | 674.49 | R | 78.7% | 2.04 | 0.79 | 5.27 | N | 6.0% |
| 20 | 2.90 | 1.77 | 4.75 | R | 62.2% | 2.79 | 0.84 | 9.28 | N | 9.5% |
| 21 | 2.86 | 1.66 | 4.93 | R | 66.6% | 2.00 | 1.11 | 3.60 | R | 18.5% |
| 22 | 0.52 | 0.30 | 0.91 | P | 19.2% | 0.36 | 0.20 | 0.62 | P | 63.8% |
| 23 | 0.45 | 0.23 | 0.90 | P | 16.7% | 0.42 | 0.26 | 0.69 | P | 50.7% |
| 24 | 0.26 | 0.09 | 0.71 | P | 54.6% | 1.15 | 0.02 | 58.59 | N | 2.7% |
| 25 | 0.16 | 0.04 | 0.74 | P | 59.1% | 1.14 | 0.02 | 58.08 | N | 2.7% |
| 26 | 0.53 | 0.31 | 0.91 | P | 27.3% | 0.11 | 0.01 | 0.86 | P | 60.0% |
| 27 | 1.33 | 0.82 | 2.17 | N | 8.7% | 0.12 | 0.02 | 0.97 | P | 53.1% |
| 28 | 0.81 | 0.50 | 1.32 | N | 6.6% | 0.09 | 0.01 | 0.69 | P | 73.2% |
| 29 | 0.77 | 0.48 | 1.23 | N | 8.3% | 0.26 | 0.09 | 0.70 | P | 55.5% |
| 30 | 5.24 | 0.10 | 282.43 | N | 5.0% | 34.23 | 1.47 | 795.56 | R | 47.0% |
| 31 | 20.16 | 1.03 | 396.19 | R | 56.1% | 0.67 | 0.33 | 1.35 | N | 4.6% |
| 32 | 19.82 | 1.01 | 389.46 | R | 55.5% | 0.69 | 0.34 | 1.41 | N | 4.4% |
| 33 | 2.84 | 1.85 | 4.35 | R | 52.3% | 3.30 | 1.08 | 10.07 | R | 8.8% |
| 34 | 2.66 | 1.70 | 4.16 | R | 58.3% | 1.56 | 0.93 | 2.64 | N | 9.3% |
| 35 | 4.06 | 1.56 | 10.54 | R | 53.5% | 2.13 | 1.40 | 3.24 | R | 37.5% |
| 36 | 0.59 | 0.36 | 0.97 | P | 12.1% | 0.40 | 0.25 | 0.63 | P | 52.4% |
| 37 | 0.58 | 0.37 | 0.92 | P | 19.5% | 0.16 | 0.03 | 0.76 | P | 50.6% |
| 38 | | | | | | | | | | |
| 39 | 21.34 | 1.15 | 394.36 | R | 68.9% | 2.19 | 0.87 | 5.49 | N | 10.9% |
| 40 | | | | | | | | | | |

In Table 1, the “‘NO:’ column provides a row number for each SNP examined in the Examples. The ‘strata’ column indicates subgroups into which subjects are classified based on the particular factors shown above, as discussed in more detail in the Examples. More specifically, ‘high age’ indicates subjects aged 55 and older and ‘low age’ indicates subjects aged younger than 55. ‘Non smoking’ indicates subjects who do not smoke and ‘smoking’ indicates subjects who smoke. ‘Male’ denotes male subjects. ‘No HTNF’ indicates subjects who do not have any hyperpiesia patients in their family history, and ‘with HTNF’ indicates subjects who have any hyperpiesia patients in their family history. ‘With HTN’ indicates subjects who have hyperpiesia and ‘w/o HTN’ indicates subjects who do not have hyperpiesia. ‘With DMF’ indicates subjects who have type II diabetes in their family history and ‘w/o DMF’ indicates subjects that do not have type II diabetes in their family history. ‘With DM’ indicates subjects who were diagnosed to have type II diabetes and ‘w/o DM’ indicates subjects who were not diagnosed to have type II diabetes. ‘High CRP’ indicates subjects who belong to the top 25% of our study population in terms of C-reactive protein (CRP) levels and ‘low CRP’ indicates subjects who belong to the bottom 25% in terms of CRP levels.

‘Alias_id’ is a SNP designation arbitrarily assigned by the inventors of the present invention.

‘SEQ ID NO:’ is the polynucleotide sequence identification number of the polymorphic sequence comprising the SNP of the present invention.

‘Gene_name’ is the symbol of the gene to which the SNP belongs. ‘Intergenic’ in this column denotes that the SNP is not located in an identified gene.

‘SNP_function’ is the role performed by the SNP deduced from the position of the known information about the gene. The designation ‘mrna_utr’ in this column denotes that the SNP is in the untranslated region of the mRNA.

‘AA_change’ indicates whether an amino acid is changed by the SNP. ‘Null’ in this column denotes that the SNP is not present in a codon, while ‘n/a’ denotes ‘not applicable’ to intergenic SNPs.

‘AA_position’ is a position in the polypeptide of an amino acid coded by the SNP site. Null’ and ‘n/a’ have the same meaning as in ‘AA_change’.

‘Cas_num’ and ‘con_num’ indicate the number of patients and normal persons of each stratum, respectively.

Allele ‘A’ and ‘a’ respectively represent a low mass allele and a high mass allele in sequencing experiments according to a homogeneous Mass EXTEND™ (hME) technique (Sequenom) and are arbitrarily designated for convenience of experiments.

‘Cas_AA’, ‘cas_Aa’ and ‘cas_aa’ respectively indicate the number of patients having the genotype ‘AA’, ‘Aa’ and ‘aa’. Also, ‘con_AA’, ‘con_Aa’ and ‘con_aa respectively indicate the number of normal persons having the genotype ‘AA’, ‘Aa’ and ‘aa’.

‘Case MAF’ and ‘control MAF’ respectively refer to the frequency of the allele ‘A’ in the patient group and the normal group.

‘Genotype Fisher's exact p-value’ is the p-value obtained from Fisher's exact test using a 3-by-2 genotype contingency table of the corresponding SNP for the patient and normal groups.

‘Allelic Fisher’s exact p-value’ is the p-value obtained from Fisher's exact test using a 2-by-2 genotype contingency table of the corresponding SNP for the patient and normal groups.

‘Allele_OR’ is the odds ratio indicating the ratio of the probability of allele_a in the patient group to the probability of allele_a in the normal group based on genotype. ‘Allele_OR_LB’ and ‘allele_OR_UB’ respectively indicate the lower limit and the upper limit of the 95% confidence interval of the odds ratio. ‘Allele_assoc’ is indicated by P (protective effect) or R (risk effect) according to whether the a allele of the corresponding SNP will have protective or risk effect on the disease compared to A allele with at a 95% significance level. ‘N’ in the ‘Allele_assoc’ column denotes nonpredictive. ‘Allele_power’ indicates the test power (1-beta risk) of the odds ratio calculation used for this genetic association study.

‘Domi_OR’ is the odds ratio indicating the ratio of the probability of the genotypes with allele_a in the patient group to the probability of genotypes with allele_a in the normal group when a dominant model is applied to the data, in which the protective or risk effect of the allele ‘a’ is exhibited in both genotypes ‘aa’ and ‘Aa’. ‘Domi_OR_LB’ and ‘domi_OR_UB’ respectively indicate the lower limit and the upper limit of the 95% confidence interval of the odds ratio using the dominant model. ‘Domi_assoc’ is indicated by P (protective effect) or R (risk effect) according to whether there is a significant association of the ‘aa’ and ‘Aa’ genotypes combined with presence of the disease at a 95% significance level. ‘Domi_power’ indicates the test power (1-beta risk) of the genetic association study using the dominant genetic model.

‘Rece_OR’ is the odds ratio of indicating the ratio of probability of the genotype aa in the patient group to the probability of the genotype aa in the normal group when a recessive model is applied to the data, in which the positive or risk effect of an allele ‘a’ is exhibited in the genotype ‘aa’. ‘Rece_OR_LB’ and ‘rece_OR_UB’ respectively indicate the lower limit and the upper limit of the 95% confidence interval of the odds ratio using the recessive model. ‘Rece_assoc’ is indicated by P (protective effect) or R (risk effect) according to whether there is a significant association of the aa genotype with the disease at a 95% significance level. ‘Rece_power’ indicates the test power (1-beta risk) of the genetic association study using the recessive genetic model.

In an embodiment of the present invention, the subjects may all be male.

In one embodiment, a polynucleotide of the present invention comprises at least 8 contiguous nucleotides of a nucleotide sequence selected from SEQ ID NO:3, SEQ ID NOS: 5 to 7 and SEQ ID NOS: 19 to 24, wherein the at least 8 contiguous nucleotides comprises a base at a SNP position in the selected nucleotide sequence. The SNPs are positioned at the 62^{nd} nucleotide in SEQ ID NO: 3, at the 77^{th} nucleotide in SEQ ID NO: 6 and at the 101^{st} nucleotide in SEQ ID NOS: 5,7 and 19 to 24. Additionally, the complement of such a polynucleotide is provided. In an embodiment of the present invention, the polynucleotide comprises 8 to 70 contiguous nucleotides of the selected nucleotide sequence.

A polynucleotide according to another aspect of the present invention is hybridized with the polynucleotide comprising the at least 8 contiguous nucleotides of a nucleotide sequence selected from SEQ ID NO: 3, SEQ ID NOS: 5 to 7 and SEQ ID NOS: 19 to 24, or the complement thereof. The polynucleotide may be allele-specific for an allele of the SNP, and may include at least 8 contiguous nucleotides, for example, 8 to 70 contiguous nucleotides.

The allele-specific polynucleotide is a polynucleotide specifically hybridized with the allele. The hybridization should be done in order to distinguish the possible alleles of the SNP sites of SEQ ID NO: 3, SEQ ID NOS: 5 to 7 and SEQ ID NOS: 19 to 24 specifically. The hybridization can be carried out under strict conditions, for example, in a salt concentration of 1 M or less and at a temperature of 25 C or higher. For example, 5×SSPE (750 mM NaCl, 50 mM Na Phosphate, 5 mM EDTA, pH 7.4) and 25 to 30° C. may be suitable conditions for the allele-specific probe hybridization.

In an embodiment of the present invention, the allele-specific polynucleotide can be a primer. The primer is a single-strand oligonucleotide capable of initiating a template-directed DNA synthesis in an appropriate buffer under appropriate conditions, for example, in the presence of four different nucleoside triphosphates and a polymerizing agent such as DNA polymerase, RNA polymerase or reverse transcriptase at a proper temperature. The length of the primer may vary according to the purpose of use, but is 15 to 30 nucleotides in an embodiment of the present invention. A short primer molecule can require a lower temperature to be stably hybridized with the template. The primer sequence does not necessarily need to be completely complementary to the template, but should be sufficiently complementary to be hybridized with the template. In an embodiment of the present invention, a 3′ end of the primer is arranged to correspond to the polymorphic site of a sequence selected from SEQ ID NO: 3, SEQ ID NOS: 5 to 7 and SEQ ID NOS: 19 to 24. The primer is hybridized with the target DNA including the polymorphic site and initiates amplification of an allele having complete homology to the primer. The primer and another primer hybridized at the opposite side are used as a primer pair for amplification of the sequence. Amplification is performed using the two primers, indicating that there is a specific allele. According to another embodiment of the present invention, the primer includes a polynucleotide fragment used in a ligase chain reaction (LCR). In an embodiment of the present invention, an allele specific polynucleotide may be a probe. The probe is a hybridization probe, which is an oligonucleotide capable of binding specifically to a complementary strand of a nucleic acid. Such a probe includes a peptide nucleic acid introduced by Nielsen et al., Science 254, 1497-1500 (1991). According to an embodiment of the present invention, the probe is an allele-specific probe. When a polymorphic site is located in DNA fragments, the allele-specific probe can hybridize with a DNA fragment having the allele to which it is complementary, but does not hybridize with a DNA fragment having the second allele to which it is not complementary. In this case, the hybridization conditions can be sufficient for hybridization with only one allele by showing a significant difference between the intensities of hybridization to the different alleles. According to an embodiment of the present invention, the probe can be arranged such that its central site is the polymorphic site of the sequence, for example the 7^{th} position in a probe consisting of 15 nucleotides, or the 8^{th} or 9^{th} position in a probe consisting of 16 nucleotides. In this way, a hybridization difference for the different alleles can be obtained. According to an embodiment of the present invention, the probe can be used for detecting an allele in a diagnosis method, etc. The diagnosis method may use, for example, Southern blotting or a microarray for detection of the allele using hybridization of the allele-specific probe.

A polypeptide according to another aspect of the present invention is encoded by the polynucleotide of SEQ ID NO: 3, SEQ ID NOS: 5 to 7 and SEQ ID NOS: 19 to 24.

An antibody according to another aspect of the present invention specifically binds to the polypeptide. The antibody may be a monoclonal antibody.

A microarray according to another aspect of the present invention comprises a polynucleotide comprising a predictive SNP of the invention. The polynucleotide comprises at least 8 contiguous nucleotides of a nucleotide sequence selected from SEQ ID Nos: 1-25, wherein the at least 8 contiguous nucleotides comprises a base at a SNP position in the selected nucleotide sequence. The SNPs are positioned at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25. In one embodiment, the nucleotide sequence is selected from SEQ ID NO: 3, SEQ ID NOS: 5 to 7 and SEQ ID NOS: 19 to 24. The microarray may also comprise the complement of the polynucleotide, a polynucleotide hybridized with one of the polynucleotides, the polypeptide encoded by one of the polynucleotides, or the cDNA thereof.

The microarray may be prepared using a conventional method known to those skilled in the art using the polynucleotide or the complement thereof, the polynucleotide hybridized with one of the poynucleotides, the polypeptide encoded by one of the polynucleotides, or the cDNA thereof.

For example, the polynucleotide may be fixed on a substrate coated with an active group of amino-silane, poly-L-lysine or aldehyde. Also, the substrate may be composed of a silicon wafer, glass, quartz, metal or plastic. The method of fixing the polynucleotide to the substrate may be a micropipetting method using piezoelectric or a method using a spotter in the shape of a pin.

A kit according to another aspect of the present invention comprises a polynucleotide comprising a predictive SNP of the invention. The polynucleotide comprises at least 8 contiguous nucleotides of a nucleotide sequence selected from SEQ ID Nos: 1-25, wherein the at least 8 contiguous nucleotides comprises a base at a SNP position in the selected nucleotide sequence. The SNPs are positioned at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25. In one embodiment, the kit comprises a polynucleotide of a nucleotide sequence selected from SEQ ID NO: 3, SEQ ID NOS: 5 to 7 and SEQ ID NOS: 19 to 24. The kit may comprise the complement of an above-mentioned polynucleotide, the polynucleotide hybridized with one of the polynucleotides, the polypeptide encoded by one of the polynucleotides, or the cDNA thereof.

The kit may further include a primer set used for isolating DNA including SNPs from diagnosed subjects or for amplifying the DNA. An appropriate primer set may be determined by those skilled in the art. For example, any of the primer sets in Table 2 may be used. Also, the kit may further include a reagent for a polymerizing reaction, for example dNTP, various polymerases and colorants.

A method according to another aspect of the present invention includes using the SNP to identify a risk of incidence of myocardial infarction for a subject.

The identifying method comprises determining an allele at a SNP. The SNP is identified by a polymorphic sequence selected from the group consisting of nucleotide sequences SEQ ID NO:1-25, wherein the SNPs are positioned at the 62^{nd} nucleotide in SEQ ID NO: 3, at the 77^{th} nucleotide in SEQ ID NO: 6, and at the 101^{st} nucleotide in SEQ ID NOS:1-2, 4-5, and 7-25. In one embodiment, the SNP is identified by a polymorphic sequence selected from the group consisting of SEQ ID NO: 3, SEQ ID NOS: 5 to 7 and SEQ ID NOS: 19 to 24. The method may further comprise obtaining a nucleic acid sample from the subject of diagnosis.

In an embodiment of the present invention, the DNA isolation may be carried out by any method known to those skilled in the art. For example, DNA can be directly purified from tissues or cells, or a specific DNA region can be amplified using a Polymerase Chain Reaction (PCR), etc. and isolated. Herein, the DNA can also be cDNA, synthesized from mRNA. Other examples of methods of obtaining nucleic acids from a subject are PCR amplification, ligase chain reaction (LCR) (Wu and Wallace, Genomics 4, 560(1989), Landegren, etc., Science 241, 1077(1988)), transcription amplification (Kwoh, etc., Proc. Natl. Acad. Sci. USA 86, 1173(1989)), self-sustained sequence replication (Guatelli, etc., Proc. Natl. Acad. Sci. USA 87, 1874(1990)) and Nucleic Acid Sequence Based Amplification (NASBA).

Determining the allele of the SNP can be determined by various methods. “Determining an allele at a SNP” can denote analyzing the allele present in the subject for one or both chromosomes. For example, sequencing the isolated DNA may be performed through various methods known to those skilled in the art to determine the allele of the snp. For example, the nucleotides of nucleic acids may be directly sequenced using a dideoxy method.

Also, the nucleotides of the polymorphic sites may be sequenced by hybridizing the DNA with a probe containing the sequence of the SNP site or a complementary probe thereof, and examining the degree of the hybridization. The degree of hybridization may be measured using a method of indicating a detectable index of the target DNA and specifically detecting the hybridized target, or using an electrical signal detecting method.

Particularly, the determining may be carried out using allele-specific probe hybridization, allele-specific amplification, homogeneous mass extension, sequencing, 5′ nuclease digestion, molecular beacon assay, oligonucleotide ligation assay, size analysis or single-stranded conformation polymorphism.

In an embodiment of the present invention, the method may further comprise judging that the subject has an increased incidence risk of myocardial infarction when the determined allele in the subject is identical to a risk allele of the SNP or judging that the subject has a lower risk of incidence of myocardial infarction when the allele determined in the subject is not a risk allele for the SNP.

According to an embodiment of the present invention, the risk allele is determined based on the relative frequencies of allele ‘A’ in the patient and normal groups. When the frequency of allele ‘A’ in the patient group is higher than that in the normal group, ‘A’ is regarded as the risk allele. In the opposite case, allele ‘a’ is regarded as the risk allele. A subject containing more risk alleles has a higher probability of having myocardial infarction.

In an embodiment of the present invention, the risk may be increased or decreased. When the frequency of an allele is higher in the normal group than in the patient group, the risk may be decreased, i.e., the allele has a protective effect against myocardial infarction. On the other hand, when the frequency of an allele of the SNP is higher in the patient group than in the normal group, the risk can be increased.

A method according to another embodiment of the present invention can identify a risk for incidence of myocardial infarction for a subject according to the subject type.

That is, the method of identifying the level of risk comprises determining an allele present in the subject at a SNP position of one or more polynucleotides among SEQ ID NOS: 1 to 25. In the method, the subject may have the following characteristics and the polynucleotides determining an allele of a polymorphic site according to the characteristics of the subject may be as follows:
a) aged 55 and older—SEQ ID NO: 1 or SEQ ID NO: 2;
b) aged 54 and younger—SEQ ID NO: 3 or SEQ ID NO: 4;
c) non-smoking—any one of SEQ ID NO: 4 to SEQ ID NO: 8;
d) male—any one of SEQ ID NO:4 and SEQ ID NO:8 to SEQ ID NO: 11;
e) not having family history of hyperpiesia—SEQ ID NO:12 or SEQ ID NO: 13;
f) having hyperpiesia—SEQ ID NO: 14;
g) not having hyperpiesia—any one of SEQ ID NO: 8 to SEQ ID NO: 10 and SEQ ID NO: 15 to SEQ ID NO: 21;
h) having family history of diabetes—SEQ ID NO: 22;
i) not having family history of diabetes—any one of SEQ ID NO: 4, SEQ ID NO: 8 to SEQ ID NO: 10, SEQ ID NO: 15, SEQ ID NO: 23 and SEQ ID NO: 24; and
j) high CRP level—SEQ ID NO: 25.

The SNPs that are predictive of the risk of myocardial infarction for a subject having a particular characteristic in the list above are identified by the polymorphic sequences following the characteristic. For example, for an individual having a high CRP level, the allele at the SNP identified by polymorphic sequence SEQ ID NO:25 should be determined for the subject, or for an individual of age 54 or younger, the allele at the SNP identified by polymorphic sequence SEQ ID NO:3 or 4 should be determined for the subject.

The method can also comprise judging that the subject has a lower risk of incidence of myocardial infarction when the allele determined in the subject is not a risk allele for the SNP; or judging that the subject has an increased risk of incidence of myocardial infarction when the allele determined in the subject is a risk allele for the SNP. The method can also include obtaining a nucleic acid sample from the subject.

A method of detecting a SNP in nucleic acid molecules according to another aspect of the present invention includes contacting a test sample containing nucleic acid molecule with a polynucleotide comprising at least 8 contiguous nucleotides of a polymorphic sequence selected from the group consisting of nucleotide sequences SEQ ID NO: 1-25, wherein the at least 8 contiguous nucleotides comprise a base at a single nucleotide polymorphism (SNP) position in the selected polymorphic sequence, wherein the SNPs are positioned at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25, or the complement of the polynucleotide, under strict hybridization conditions such that specific hybridization between nucleic acid molecules in the test sample and the polynucleotide can occur; and detecting formation of a hybridized double-strand. In some embodiments, the polymorphic sequence is selected from the group consisting of SEQ ID NO: 3, SEQ ID NOS: 5 to 7 and SEQ ID NOS: 19 to 24.

The detecting method may be carried out using allele-specific probe hybridization, allele-specific amplification, sequencing, 5′ nuclease digestion, molecular beacon assay, oligonucleotide ligation assay, size analysis and single-stranded conformation polymorphism method.

A method of screening pharmaceutical compositions for myocardial infarction according to another aspect of the present invention includes contacting a candidate material with a polypeptide encoded by the polynucleotide comprising at least 8 contiguous nucleotides of a nucleotide sequence selected from SEQ ID NO:3, SEQ ID NOS: 5 to 7 and SEQ ID NOS: 19 to 24, wherein the at least 8 contiguous nucleotides comprises a base at a SNP position in the selected nucleotide sequence under proper conditions for formation of a binding complex; and detecting formation of the binding complex between the polypeptide and the candidate material.

The screening method may be carried out though coimmunoprecipitation, Radioimmunoassay (RIA), Enzyme Linked ImmunoSorbent Assay (ELISA), Immunohistochemistry, Western Blotting and Fluorescence Activated Cell Sorting (FACS).

The present invention will now be described in greater detail with reference to the following examples. The following examples are for illustrative purposes only and are not intended to limit the scope of the invention.

### EXAMPLE 1

SNP Selection

DNA was isolated from blood of a patient group diagnosed with and being treated for a cardiovascular disease and also from blood of a normal group not having symptoms of cardiovascular disease. Appearance frequency of the alleles of a specific SNP was then analyzed. The SNPs in the Example of the present invention were selected from either a public database (NCBI dbSNP, available at http://www.ncbi.nim.nih.gov/SNP/) or a commercial database available from Sequenom (http:://www.realsnp.com/). The SNPs were analyzed using a primer hybridizing close to the selected SNP.

1-1. Preparation of DNA Sample

DNA was extracted from blood of a patient group including 221 Korean male patients diagnosed with and being treated for myocardial infarction and also from a normal group including 192 Korean men not having myocardial infarction. The DNA extraction was carried out according to a known extraction method (Molecular cloning: A Laboratory Manual, p 392, Sambrook, Fritsch and Maniatis, 2nd edition, Cold Spring Harbor Press, 1989) and guidelines of a commercially available kit (Gentra system, D-50K). Only DNA having purity of at least 1.7, measured using UV (260/280 nm), was selected from the extracted DNA and used.

1-2. Amplification of the Target DNA

The target DNA having a certain DNA region including a SNP to be analyzed was amplified using a PCR. The PCR was performed using a general method and conditions as indicated below. 2.5 ng/ml of the target genome DNA was first prepared. Then, the following PCR reaction solution was prepared.

| | | |
|---|---|---|
| Water (HPLC grade) | 3.14 | μl |
| 10×buffer | 0.5 | μl |
| MgCl₂ (25 mM) | 0.2 | μl |
| dNTP mix (GIBCO)(25 mM/each) | 0.04 | μl |
| Taq pol (HotStart)(5 U/μl) | 0.02 | μl |
| Forward/reverse primer mix (10 μM) | 0.1 | μl |
| DNA | 1.00 | μl |
| Total reaction volume | 5.00 | μl |

The forward and reverse primers were selected to hybridize upstream and downstream from the SNP at suitable positions. The primer set for each SNP is listed in Table 2.

Thermal cycling of PCR was performed by maintaining the temperature at 95° C. for 15 minutes, cycling the temperature from 95° C. for 30 seconds, to 56° C. for 30 seconds and to 72° C. for 1 minute a total of 45 times, maintaining the temperature at 72° C. for 3 minutes, and then storing at 4° C. As a result, target DNA fragments having 200 nucleotides or less were obtained.

1-3. Analysis of SNP of the Amplified Target DNA

Analysis of the SNP of the target DNA fragment was performed using the homogeneous MassEXTEND (hME) technique from Sequenom. The principle of the hME is as follows. First, a primer, also called an extension primer, complementary to bases up to just before the SNP of the target DNA fragment is prepared. The primer is hybridized with the target DNA fragment and DNA polymerization is facilitated. At this time, added to the reaction solution is a reagent (Termination mix, e.g. ddTTP) for terminating polymerization after the base complementary to a first allele base (e.g. ‘A’ allele) among the two alleles of the subject SNP was added to the polymerizing chain. As a result, if the target DNA fragment includes the first allele (e.g. ‘A’ allele), a product is obtained having only one base complementary to the first allele (e.g. ‘T’) added. On the other hand, if the target DNA fragment includes a second allele (e.g. ‘G’ allele), a product is obtained having a base complementary to the second allele (e.g. ‘C’) and extending to the nearest base identical to the first allele base (e.g. ‘A’). The length of the product extending from the primer is determined using mass analysis to determine the type of allele in the target DNA.

Specific experimental conditions for the analysis of the SNPs listed in Table 1 were as follows. First, free dNTPs were removed from the PCR product. To this end, 1.53 μl of pure water, 0.17 μl of an hME buffer, and 0.30 μl of shrimp alkaline phosphatase (SAP) were added to a 1.5 ml tube and mixed to prepare a SAP enzyme solution. The tube was centrifuged at 5,000 rpm for 10 seconds. Then, the PCR product was put into the SAP solution tube, sealed, maintained at 37° C. for 20 minutes and at 85° C. for 5 minutes, and then stored at 4° C.

Next, a homogeneous extension reaction was performed using the target DNA product as a template. The reaction solution was as follows.

| | | |
|---|---|---|
| Water (nanopure grade) | 1.728 | μl |
| hME extension mix | 0.200 | μl |
| (10×buffer containing 2.25 mM d/ddNTPs) | | |
| Extension primer (each 100 μM) | 0.054 | μl |
| Thermosequenase (32 U/μl) | 0.018 | μl |
| Total volume | 2.00 | μl |

The reaction solution was mixed well and spin down centrifuged. A tube or plate containing the reaction solution was sealed and maintained at 94° C. for 2 minutes, cycled from 94° C. for 5 seconds, to 52° C. for 5 seconds to 72° C. for 5 seconds a total of 40 times, and then stored at 4° C. The obtained homogeneous extension product was washed with a resin (SpectroCLEAN, Sequenom, #10053) and salt was removed. The extension primers used for homogeneous extension for each SNP are indicated in Table 2.

TABLE 2

| | Primer for target | | |
|---|---|---|---|
| Nucleotide including | DNA amplification | | Extension |
| SNP | (SEQ ID NO:) | | primer |
| (SEQ ID NO:) | Forward primer | Reverse primer | (SEQ ID NO:) |
| 1 | 26 | 27 | 28 |
| 2 | 29 | 30 | 31 |
| 3 | 32 | 33 | 34 |
| 4 | 35 | 36 | 37 |
| 5 | 38 | 39 | 40 |
| 6 | 41 | 42 | 43 |
| 7 | 44 | 45 | 46 |
| 8 | 47 | 48 | 49 |
| 9 | 50 | 51 | 52 |
| 10 | 53 | 54 | 55 |
| 11 | 56 | 57 | 58 |
| 12 | 59 | 60 | 61 |
| 13 | 62 | 63 | 64 |
| 14 | 65 | 66 | 67 |
| 15 | 68 | 69 | 70 |
| 16 | 71 | 72 | 73 |
| 17 | 74 | 75 | 76 |
| 18 | 77 | 78 | 79 |
| 19 | 80 | 81 | 82 |
| 20 | 83 | 84 | 85 |
| 21 | 86 | 87 | 88 |
| 22 | 89 | 90 | 91 |
| 23 | 92 | 93 | 94 |
| 24 | 95 | 96 | 97 |
| 25 | 98 | 99 | 100 |

Mass analysis was performed on the extension products to determine the allele of the polymorphic site using Matrix Assisted Laser Desorption and Ionization-Time of Flight (MALDI-TOF). In the MALDI-TOF, a material to be analyzed is exposed to a laser beam and flies with an ionized matrix (3-Hydroxypicolinic acid) in a vacuum to a detector. The flight time to the detector is calculated to determine the mass. A light material can reach the detector in a shorter amount of time than a heavy material. The nucleotide sequences of SNPs in the target DNA were determined based on differences in mass and known nucleotide sequences of the SNPS.

1-4. Selection of SNP

The patient group and the normal group were each divided into a high risk group and a low risk group in consideration of degree of risk due to environmental or habitual factors. That is, the patient group was divided into a high risk patient group and a low risk patient group and the normal group was divided into a high risk normal group and a low risk normal group.

Specifically, the factors dividing the subjects into the high risk group and the low risk group were age (55 or older vs. younger than 55), smoking, hyperpiesia, family history of hyperpiesia, diabetes, family history of diabetes, top 25% and bottom 25% c-reactive protein (CRP) level, top 25% and bottom 25% low density lipoprotein (LDL) level; and top 25% and bottom 25% total cholesterol level.

The allele frequencies in the patient group and the normal group, the high risk patient group and the high risk normal group, and the low risk patient group and the low risk normal group were each compared using the Fisher's exact test as an association test.

The effect size was determined using an allele odds ratio and the 95% confidence interval thereof. When the normal group had a higher frequency of a given allele than the patient group, the allele was associated with decreased risk of myocardial infarction and the other allele was determined to be the risk allele for myocardial infarction. On the other hand, when the patient group had a higher frequency of a given allele than the normal group, that allele was determined to be the risk allele.

For a SNP for which the Fisher's exact test p-value was less than 0.05 and the 95% confidence interval of the odds ratio value with the strongest association among those of three 2-by-2 contingency tables, base on allele frequency, dominant genetic model or recessive genetic model respectively, does not include 1.0 if the power of the test is larger than 50%.

The results are listed in Table 1. As shown in Table 1, 25 SNPs associated with myocardial infarction were identified in a Korean population.

### EXAMPLE 2

Preparation of SNP Immobilized Microarray

A microarray is prepared by immobilizing nucleic acids comprising the selected SNPs on a substrate. That is, polynucleotides are immobilized on the substrate, wherein each polynucleotide comprises 20 contiguous nucleotides of a nucleotide sequence selected from SEQ ID NOS: 1 to 25, wherein the 20 contiguous nucleotides comprise the polymorphism of the selected sequence located at the 11^{th} base of the 20 contiguous nucleotides.

First, 5′-ends of each of the polynucleotides are substituted with an amine group and the polynucleotides are spotted onto a silylated slide (Telechem) using 2×SSC (0.3M NaCl, 0.03M sodium citrate, 0.1 mM EDTA, pH 7.0), as a spotting buffer. After the spotting, binding is induced in a drying machine and free oligonucleotides are removed by washing with sodium dodecyl sulphate (SDS) for 2 minutes and with triple distilled water for 2 minutes. The microarray is prepared using denaturation induced by increasing the temperature of the slide to 95° C. for 2 minutes, washing with a blocking solution (1.0 g NaBH₄, phosphate buffered saline (PBS) (pH 7.4) 300 mL, 100 mL ethanol) for 15 minutes, a 0.2% SDS solution for 1 minute and triple distilled water for 2 minutes, and then drying at room temperature.

### EXAMPLE 3

Diagnosis of Myocardial Infarction Using the Mycroarray

DNA is isolated from blood of a subject for diagnosis of the incidence or risk of myocardial infarction. The target DNA is labeled with a fluorescent material using the methods described in Examples 1-1 and 1-2. The fluorescent labeled target DNA is hybridized with the microarray of Example 2 at 42° C. for 4 hours in a UniHyb hybridization solution (TeleChem). The slide is washed twice with 2×SSC at room temperature for 5 minutes and dried in air. The dried slide is scanned using a ScanArray (GSI Lumonics). The scanned results are analyzed using a QuantArray (GSI Lumonics) and ImaGene software (BioDiscover). The probability of incidence of myocardial infarction and the susceptibility thereto are measured by identifying whether the subject had the SNP according to an embodiment of the present invention.

The SNPs of the present invention associated with myocardial infarction in a Korean population can be used for diagnosis and treatment of myocardial infarction and gene fingerprinting analysis. By using the microarray and the kit including the SNPs of the present invention, the presence or risk of myocardial infarction can be effectively diagnosed according to the type of subject. Similarly, by using the method of analyzing the SNPs associated with myocardial infarction of the present invention, the presence or risk of myocardial infarction can effectively be diagnosed according to the type of subject.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

While the present invention has been particularly shown and described with reference to exemplary embodiments thereof, it will be understood by those of ordinary skilled in the art that various changes in form and details may be made therein without departing from the spirit and scope of the present invention as defined by the following claims. Moreover, any combination of the above-described elements in all possible variations thereof is encompassed by the invention unless otherwise indicated herein or otherwise clearly contradicted by context.

What is claimed is:

1. A polynucleotide comprising:
(a) a nucleic acid comprising at least 8 contiguous nucleotides of a polymorphic sequence selected from the group consisting of nucleotide sequences SEQ ID NO: 3, SEQ ID NOS: 5 to 7 and SEQ ID NOS: 19 to 24, wherein the at least 8 contiguous nucleotides comprise a base at a single nucleotide polymorphism (SNP) position in the selected polymorphic sequence, wherein the SNPs are positioned at the 62^{nd} nucleotide in SEQ ID NO: 3, at the 77^{th} nucleotide in SEQ ID NO: 6 and at the 101^{st} nucleotide in SEQ ID NOS: 5, 7 and 19 to 24; or
(b) the complement of (a).

2. The polynucleotide of claim 1, comprising 8 to 70 contiguous nucleotides of the selected polymorphic sequence, or the complement thereof.

3. A polynucleotide that specifically hybridizes with the polynucleotide of claim 1.

4. The polynucleotide of claim 3, comprising 8 to 70 nucleotides.

5. The polynucleotide of claim 3, wherein the polynucleotide is an allele specific probe.

6. The polynucleotide of claim 3, wherein the polynucleotide is an allele specific primer.

7. A polypeptide encoded by the polynucleotide of claim 1.

8. An antibody, wherein the antibody binds specifically to the polypeptide of claim 7.

9. The antibody of claim 8, wherein the antibody is a monoclonal antibody.

10. A microarray for detecting a SNP comprising
the polynucleotide of claim 1, a polypeptide encoded by the polynucleotide of claim 1, or a cDNA thereof.

11. A kit for detecting a SNP comprising
the polynucleotide of claim 1, a polypeptide encoded by the polynucleotide of claim 1, or a cDNA thereof.

12. A method of identifying a risk of incidence of myocardial infarction for a subject, the method comprising:
determining an allele present in the subject at a SNP,
wherein the SNP is identified by a polymorphic sequence selected from the group consisting of SEQ ID NO:1-25,
wherein the SNPs are positioned at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25.

13. The method of claim 12, wherein the SNP is identified by a polymorphic sequence selected from the group consisting of SEQ ID NO: 3, SEQ ID NOS: 5 to 7 and SEQ ID NOS: 19 to 24.

14. The method of claim 12, further comprising:
obtaining a nucleic acid sample from the subject.

15. The method of claim 12,
wherein determining the allele is carried out by performing a method selected from the group consisting of allele-specific probe hybridization, allele-specific amplification, homogeneous mass extension, sequencing, 5′ nuclease digestion, molecular beacon assay, oligonucleotide ligation assay, size analysis and single-stranded conformation polymorphism.

16. The method of claim 12, further comprising:
judging that the subject has a lower risk of incidence of myocardial infarction when the allele determined in the subject is not a risk allele for the SNP.

17. The method of claim 12, further comprising:
judging that the subject has an increased risk of incidence of myocardial infarction when the allele determined in the subject is a risk allele for the SNP.

18. A method of identifying risk of incidence of myocardial infarction for a subject, the method comprising:
determining an allele present in the subject at a SNP,
wherein if the subject is aged 55 and older, then the SNP is identified by a polymorphic sequence SEQ ID NO: 1 or SEQ ID NO: 2;
wherein if the subject is aged 54 and younger, then the SNP is identified by a polymorphic sequence SEQ ID NO: 3 or SEQ ID NO: 4;
wherein if the subject is non-smoking, then the SNP is identified by a polymorphic sequence selected from the group consisting of SEQ ID NO: 4 to SEQ ID NO: 8;
wherein if the subject is male, then the SNP is identified by a polymorphic sequence selected from the group consisting of SEQ ID NO: 4 and SEQ ID NO: 8 to SEQ ID NO:11;
wherein if the subject does not have a family history of hyperpiesia, then the SNP is identified by a polymorphic sequence SEQ ID NO: 12 or SEQ ID NO: 13;
wherein if the subject has hyperpiesia, then the SNP is identified by polymorphic sequence SEQ ID NO: 14;
wherein if the subject does not have hyperpiesia, then the SNP is identified by a polymorphic sequence selected from the group consisting of SEQ ID NO: 8 to SEQ ID NO: 10 and SEQ ID NO: 5 to SEQ ID NO: 21;
wherein if the subject has a family history of diabetes, then the SNP is identified by polymorphic sequence SEQ ID NO: 22;
wherein if the subject does not have a family history of diabetes, then the SNP is identified by a polymorphic sequence selected from the group consisting of SEQ ID NO: 4, SEQ ID NO: 8 to SEQ ID NO: 10, SEQ ID NO: 23 and SEQ ID NO: 24; or
wherein if the subject has a high CRP level, then the SNP is identified by polymorphic sequence SEQ ID NO: 25;
wherein the SNPs are positioned at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25.

19. The method of claim 18, further comprising
obtaining a nucleic acid sample from the subject.

20. The method of claim 18, further comprising:
judging that the subject has a lower risk of incidence of myocardial infarction when the allele determined in the subject is not a risk allele for the SNP; or.
judging that the subject has an increased risk of incidence of myocardial infarction when the allele determined in the subject is a risk allele for the SNP.

21. A method of detecting a SNP in nucleic acid molecules, the method comprising:
contacting a test sample containing nucleic acid molecules with a polynucleotide comprising at least 8 contiguous nucleotides of a polymorphic sequence selected from the group consisting of nucleotide sequences SEQ ID NO: 1-25, wherein the at least 8 contiguous nucleotides comprise a base at a single nucleotide polymorphism (SNP) position in the selected polymorphic sequence, wherein the SNPs are positioned at the 62^{nd} nucleotide in SEQ ID NO: 3, at the 77^{th} nucleotide in SEQ ID NO: 6 and at the 101^{st} nucleotide in SEQ ID NOS:1-2, 4-5, and 7-25, or the complement thereof, under strict hybridization conditions such that specific hybridization between nucleic acid molecules in the test sample and the polynucleotide can occur; and
detecting the formation of a hybridized double-strand.

22. The method of claim 21,
wherein the detecting the formation of a hybridized double-strand is carried out by performing a method selected from the group consisting of allele-specific probe hybridization, allele-specific amplification, sequencing, 5′ nuclease digestion, molecular beacon assay, oligonucleotide ligation assay, size analysis and single-stranded conformation polymorphism.

23. The method of claim 21, wherein the polymorphic sequence is selected from the group consisting of SEQ ID NO: 3, SEQ ID NOS: 5 to 7 and SEQ ID NOS: 19 to 24.

24. A method of screening pharmaceutical compositions for myocardial infarction, the method comprising:
contacting a candidate material with the polypeptide of claim 7 under suitable conditions for formation of a binding complex; and
detecting formation of the binding complex between the polypeptide and the candidate material.

25. The method of claim 24, wherein detecting formation of the binding complex is carried out by performing a method selected from the group consisting of coimmunoprecipitation, Radioimmunoassay (RIA), Enzyme Linked ImmunoSorbent Assay (ELISA), Immunohistochemistry, Western Blotting and Fluorescence Activated Cell Sorting (FACS).
